(19) United States
(12) Patent Application Publication
(10) Pub. No.: US 2011/0308605 A1
(43) Pub. Date: Dec. 22, 2011

(54) Applicable Fluorescence of Diamondoids

(75) Inventors: Zhi Liu, Millbrae, CA (US); Will Clay, Stanford, CA (US); Michael A. Kelly, Portola Valley, CA (US); Nicholas A. Melosh, Menlo Park, CA (US); Zhi-Xun Shen, Stanford, CA (US); Andrey A. Fokin, Giessen (DE); Peter R. Schreiner, Wettenberg (DE)

(73) Assignees: Justus-Liebig-Universitaet Giessen, Giessen (DE); Leland J. Stanford Junior Univesity, Palo Alto, CA (US)

(21) Appl. No.: 12/865,390

(22) PCT Filed: Jan. 30, 2009
(86) PCT No.: PCT/US2009/000618

§ 371 Date: Jan. 21, 2011

Related U.S. Application Data
(60) Provisional application No. 61/006,800, filed on Jan. 31, 2008.

(51) Int. Cl. H01L 31/0216; C07C 13/615; H05B 33/14

(52) U.S. Cl. 136/257; 313/503; 585/22

(57) ABSTRACT

Provided is a fluorescent diamondoid material which when energized, either by an electric field or by high energy radiation, emits light. The light emitted is generally in the visible range. The diamondoid material can be fine tuned by internal or external doping. The fluorescent materials comprised of diamondoids, have applications in several fields. One application is in solar cells where these materials can be used to improve the overall efficiency of the device. A second application is in indoor lighting where the materials can be used to efficiently produce white light. This can be done by either using the material as a fluorescent medium for a UV light source, in an electroluminescence device, or by using the material as part of an organic light emitting diode (OLED).

### BACKGROUND

Fluorescence is a type of luminescence, and is generally an optical phenomenon in which the molecular absorption of a photon triggers the emission of another photon at a different wavelength, generally a longer wavelength. Usually the absorbed photon is in the ultraviolet range, and the emitted light is in the visible range. Fluorescence can also occur when a molecule relaxes to its ground state after being electronically excited. There are many natural and synthetic compounds that exhibit fluorescence, and they have a number of applications. The most prominent is that of lighting.

The common fluorescent tube relies on fluorescence. Inside the glass tube is a partial vacuum and a small amount of mercury. An electric discharge in the tube causes the mercury atoms to emit light. The emitted light is in the ultraviolet range. The tube is generally lined with a coating of a fluorescent material or phosphor, which absorbs the ultraviolet light and emits visible light.

Fluorescence is a phenomenon that can lend itself to many different applications, and the search for novel and appropriate fluorescent materials is ongoing. Providing such a novel fluorescent material which can be utilized and improve the efficiency of fluorescent applications is a present objective.

### SUMMARY

Provided is a fluorescent diamondoid material which when energized, either by an electric field or by high energy radiation, emits light. The light emitted is generally in the visible range. The diamondoid material can be fine tuned in its light emission by internal or external doping, by making derivatives with different energy gaps.

The fluorescence observed when the diamondoid material is energized permits its use in many applications. Of particular note is a solar cell and in lighting. Among other factors, the use of an energized diamond film has been found to be most effective in emitting visible light. The ability to fine tune the fluorescence of the diamondoid material also allows one to maximize effectiveness. It is also believed that the use of diamondoid materials can provide multiphoton generation under the proper circumstances, which can be controlled. Such multiphoton generation permits maximum efficiency in applications such as solar cells and lighting

### BRIEF DESCRIPTION OF THE FIGURES

FIG. 1(a) and FIG. 1(b) illustrate two embodiments of a solar cell using fluorescent diamondoid film.

FIG. 2 depicts the conversion of one high energy photon, in this case a UV photon, into more than one visible photon.

FIG. 3 shows the fluorescence spectra from adamantane, diamantane and triamantane under 229 nm UV illumination.

FIG. 4 shows the fluorescence spectra from tetramantane and pentamantane under 229 nm UV illumination.

FIG. 5 shows a side view of one embodiment of a solar cell having a top electrode with openings.

FIG. 6 shows a top view of one embodiment of a solar cell having a top electrode with openings.

### DETAILED DESCRIPTION

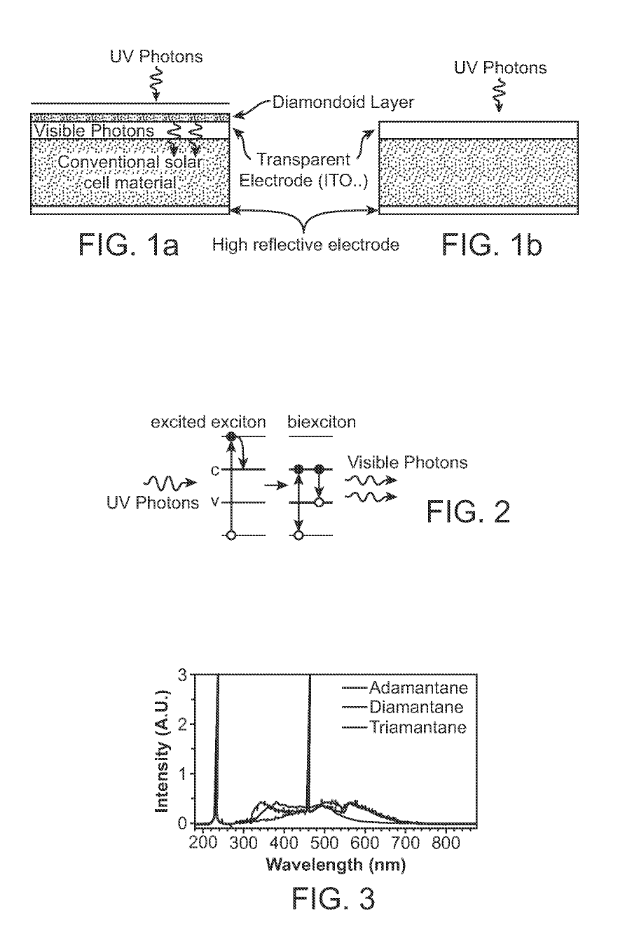

Provided are novel fluorescent materials comprised of diamondoids, which have applications in several fields. One application is in solar cells where these materials can be used to improve the overall efficiency of the device. A solar cell can be modified in several ways (as shown in FIG. 1) to include these materials, which are nanostructures of carbon (known as diamondoids). The goal of these modifications is to increase the efficiency of collecting high energy photons (those including near UV radiation in the solar spectrum with more than twice the band gap of typical solar cell materials such as Si), by converting each UV photon into one or more visible photons, which can be captured more efficiently in the cell. This can be achieved by either coating the solar cell with a thin film of the material (FIG. 1a) or by embedding small crystals of the material within the semiconductor itself (FIG. 1b).

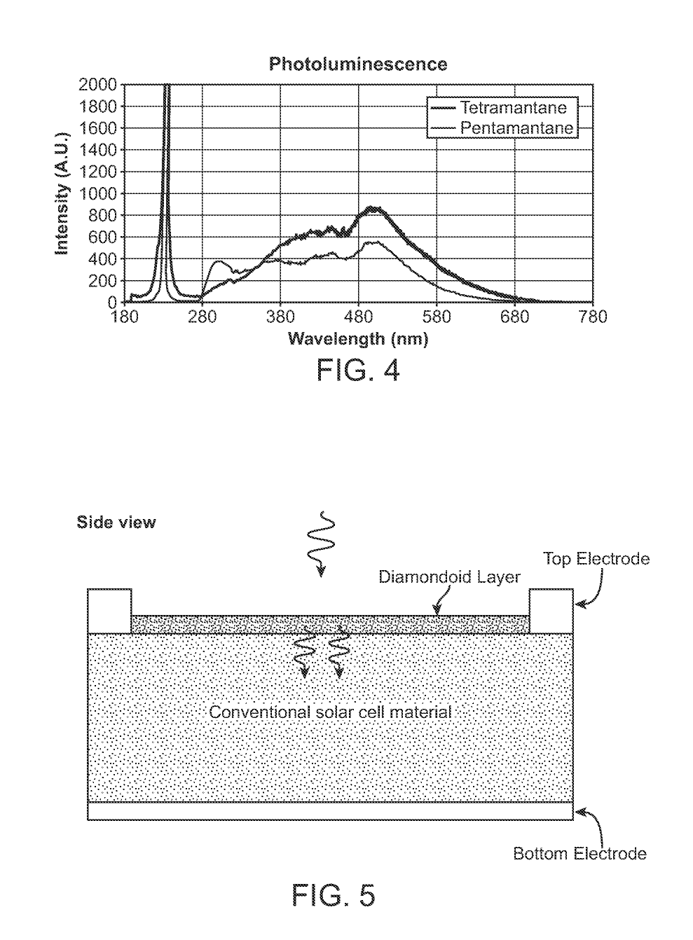

A second important application is in lighting where the materials can be used to efficiently produce white light, or other light color for a desired lighting application, for example, indoor lighting design. This can be done by either using the material as a fluorescent medium for a UV light source, by using the material as part of an organic light emitting diode (OLED), or through an electroluminescence device. When relevant, all processes should enable the efficient production of light due to the material's ability to convert one high energy photon or exciton into several lower energy (visible) photons. Another major advantage of these materials over other fluorescent media is the nearly white emission spectrum they produce (as shown in FIGS. 3 and 4).

According to embodiments of the present invention diamondoids are isolated from an appropriate feedstock, and then fabricated into a material or film useful in an environment, e.g., in a solar cell or lighting system, in which it will be energized and then effectively emit light. The diamondoid can be energized by electric current or high energy radiation

The term “diamondoids” refers to substituted and unsubstituted cage compounds of the adamantane series including adamantane, diamantane, triamantane, tetramantanes, pentamantanes, hexamantanes, heptamantanes, octamantanes, nonamantanes, decamantanes, undecamantanes, and the like, including all isomers and stereoisomers thereof. The compounds have a “diamondoid” topology, which means their carbon atom arrangement is superimposable on a fragment of a FCC diamond lattice. Substituted diamondoids from the first 10 of the series are preferable with 1 to 4 independently-selected alkyl substituents. Diamondoids include “lower diamondoids” and “higher diamondoids,” as these terms are defined herein, as well as mixtures of any combination of lower and higher diamondoids.

The term “lower diamondoids” refers to adamantane, diamantane and triamantane and any and/or all unsubstituted and substituted derivatives of adamantane, diamantane and triamantane. These lower diamondoid components show no isomers or chirality and are readily synthesized, distinguishing them from “higher diamondoids.”

The term “higher diamondoids” refers to any and/or all substituted and unsubstituted tetramantane components; to any and/or all substituted and unsubstituted pentamantane components; to any and/or all substituted and unsubstituted hexamantane components; to any and/or all substituted and unsubstituted heptamantane components; to any and/or all substituted and unsubstituted nonamantane components; to any and/or all substituted and unsubstituted decamantane components; to any and or all substituted and undecamantane components; as well as mixtures of the above and isomers and stereoisomers of tetramantane, pentamantane, hexamantane, heptamantane, octamantane, nonamantane, decamantane, and undecamantane.

Adamantane chemistry has been reviewed by Fort, Jr. et al. in “Adamantane: Consequences of the Diamondoid Structure,” Chem. Rev. vol. 64, pp. 277-300 (1964). Adamantane is the smallest member of the diamondoid series and may be thought of as a single cage crystalline subunit. Diamantane contains two subunits, triamantane three, tetramantane four, and so on. While there is only one isomeric form of adamantane, diamantane, and triamantane, there are four different isomers of tetramantane, (two of which represent an enantiomeric pair), i.e., four different possible ways or arranging the four adamantane subunits. The number of possible isomers increases non-linearly with each higher member of the diamondoid series, pentamantane, hexamantane, heptamantane, octamantane, nonamantane, decamantane, etc.

Adamantane, which is commercially available, has been studied extensively. The studies have been directed toward a number of areas, such as thermodynamic stability, functionalization, and the properties of adamantane-containing materials. For instance, the following patents discuss materials comprising adamantane subunits: U.S. Pat. No. 3,457,318 teaches the preparation of polymers from alkenyl adamantanes; U.S. Pat. No. 3,832,332 teaches a polyamide polymer forms from alkyladamantane diamine; U.S. Pat. No. 5,017,734 discusses the formation of thermally stable resins from adamantane derivatives; and U.S. Pat. No. 6,325,851 reports the synthesis and polymerization of a variety of adamantane derivatives.

The four tetramantane structures are iso-tetramantane [1(2)3], anti-tetramantane [121] and two enantiomers of skew-tetramantane [123], with the bracketed nomenclature for these diamondoids in accordance with a convention established by Balaban et al. in “Systematic Classification and Nomenclature of Diamond Hydrocarbons-I,” Tetrahedron vol. 34, pp. 3599-3606 (1978). All four tetramantanes have the formula C₂₂H₂₈ (molecular weight 292). There are ten possible pentamantanes, nine having the molecular formula C₂₆H₃₂ (molecular weight 344) and among these nine there are three pairs of enantiomers represented generally by [12(1)3)], [1234], [1213] with the nine enantiomeric pentamantanes represented by [12(3)4], [1212]. There also exists a pentamantane [1231] represented by the molecular formula C₂₅H₃₀ (molecular weight 330).

Hexamantanes exist in thirty-nine possible structures with twenty eight having the molecular formula C₃₀H₃₆ (molecular weight 396) and of these, six are symmetrical; ten hexamantanes have the molecular formula C₂₉H₃₄ (molecular weight 382) and the remaining hexamantane [12312] has the molecular formula C₂₆H₃₀ (molecular weight 342).

Heptamantanes are postulated to exist in 160 possible structures with 85 having the molecular formula C₃₄H₄₀ (molecular weight 448) and of these, seven are achiral, having no enantiomers. Of the remaining heptamantanes, 67 have the molecular formula C₃₃H₃₈ (molecular weight 434), six have the molecular formula C₃₂H₃₆ (molecular weight 420) and the remaining two have the molecular formula C₃₀H₃₄ (molecular weight 394).

Octamantanes possess eight of the adamantane subunits and exist with five different molecular weights. Among the octamantanes, 18 have the molecular formula C₄₃H₃₈ (molecular weight 446). Octamantanes also have the molecular formula C₃₈H₄₄ (molecular weight 500); C₃₇H₄₂ (molecular weight 486); C₃₆H₄₀ (molecular weight 472), and C₃₃H₃₆ (molecular weight 432).

Nonamantanes exist within six families of different molecular weights having the following molecular formulas; C₄₂H₄₈ (molecular weight 552), C₄₁H₄₆ (molecular weight 538), C₄₀H₄₄ (molecular weight 524), C₃₈H₄₂ (molecular weight 498), C₃₇H₄₀ (molecular weight 484) and C₃₄H₃₆ (molecular weight 444).

Decamantane exists within families of seven different molecular weights. Among the decamantanes, there is a single decamantane having the molecular formula C₃₅H₃₆ (molecular weight 456) which is structurally compact in relation to the other decamantanes. The other decamantane families have the molecular formulas: C₄₆H₅₂ (molecular weight 604); C₄₅H₅₀ (molecular weight 590); C₄₄H₄₈ (molecular weight 576); C₄₂H₄₆ (molecular weight 550); C₄₁H₄₄ (molecular weight 536); and C₃₈H₄₀ (molecular weight 496).

Undecamantane exists within families of eight different molecular weights. Among the undecamantanes there are two undecamantanes having the molecular formula C₃₉H₄₀ (molecular weight 508) which are structurally compact in relation to the undecamantanes. The other undecamantane families have the molecular formulas C₄₁H₄₂ (molecular weight 534); C₄₂H₄₄ (molecular weight 548); C₄₅H₄₈ (molecular weight 588); C₄₆H₅₀ (molecular weight 602); C₄₈H₅₂ (molecular weight 628); C₄₉H₅₄ (molecular weight 642); and C₅₀H₅₆ (molecular weight 656).

Methods of forming diamondoid derivatives, heterodiamondoids, and polymerizing diamondoids, are discussed, for example, in U.S. Pat. No. 7,049,344; U.S. Patent Publication 2003/0193710; and U.S. Patent Publication 2002/0177743; which are all incorporated herein by reference in their entirety to an extent not inconsistent herewith.

Turning to FIG. 1, the embodiment in FIG. 1(a) shows a solar cell comprised of a lower layer which is an electrode. The lower electrode can be highly reflective in one embodiment. Above the electrode is a layer of semiconductive solar cell material. This layer can be any suitable semiconductor material useful in solar cells, as is known in the industry. The electrode and semiconductor material accumulates the photon energy and converts same to electricity. Above the layer of solar cell material is a transparent electrode or electrode with openings, in order to allow the light photons passage to the solar cell material. A diamondoid layer is coated on the transparent layer or in the openings. The diamondoid layer faces the photon source e.g., the sun, and is excited by the high energy photons of the solar spectrum, e.g., UV energy. The diamondoid film then emits visible photons, which can be more than one for every high energy photon, e.g., UV, near UV or deep blue photon, through the transparent electrode and into the semiconductor material. This re-emitting process has the added benefit of changing the light propagation direction which can be tuned to increase the absorption efficiency by the solar cell. The diamondoid layer is generally of sufficient thickness to absorb all of the UV sunlight. In another embodiment, an anti-reflective coating is coated over the diamondoid film. This coating ensures that all the photons are directed into the solar cell and are not lost by reflection.

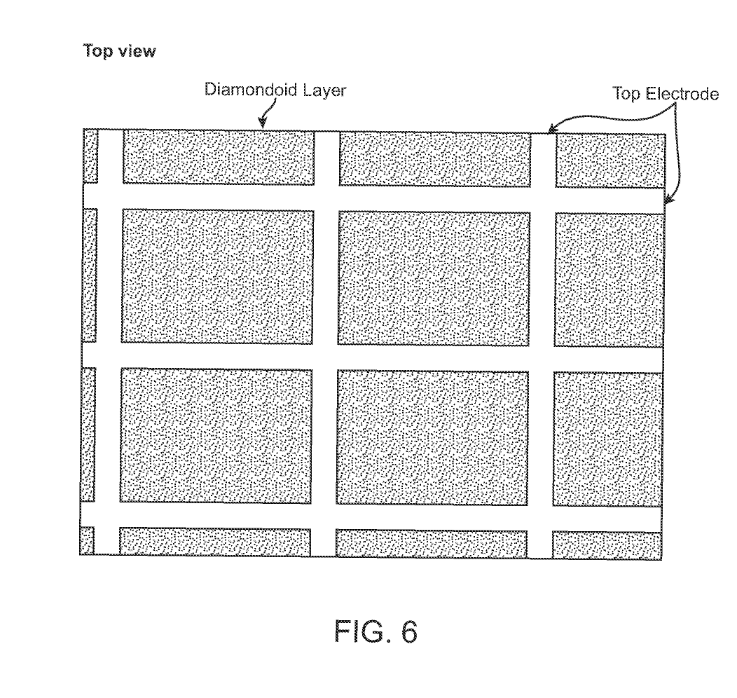

The embodiment shown in FIGS. 5 and 6 depicts one example of the electrode above the solar material having openings and with the diamondoid layer being coated in the openings. FIG. 5 shows a sideview. FIG. 6 shows a top view with the diamondoid material in the openings.

Another embodiment is shown in FIG. 1(b). The solar cell has a lower electrode layer, which can be highly reflective. Above the electrode is the semiconductor material, which also contains some diamondoid material. The diamondoid material is dispersed in the semiconductor material and absorbs the UV energy, becomes excited, and re-emits visible photons. The top layer can be a transparent electrode or an electrode with openings.

In another embodiment, a phosphor layer is present over the diamondoid layer. The phosphor layer is present to absorb the UV energy and re-emit light at a wavelength that is fine tuned to the absorption spectrum of the diamondoid layer. The efficiency of the system is therefore greatly increased.

In general, the working principle is that nanocrystal materials have a huge advantage over bulk materials in multi-exciton generation (100% vs. <1%), meaning one high energy photon can generate 2 or even 3 low energy excitons. Thus, it is believed that multiple low energy photons can be created through the recombination of these low energy excitons and one UV photon can be converted to more than one visible photons. This is shown in FIG. 2.

While pure triamantane crystals show fluorescence upon irradiation with high-energy light it is clear that functional groups (external doping) and/or heterosubstitution (internal doping) can further improve efficiency by tuning the fluorescence shifts and by providing attachment points for diamondoid assemblies on surfaces or for use in a variety of materials (polymers etc.). It is likely that the fluorescence properties are related to the HOMO-LUMO gap (energy separation) and the polarization as well as polarizability of externally and/or internally functionalized diamondoids. This can be achieved by selective chemical methods to incorporate functional groups to replace C—H-bonds in well-defined positions on the diamondoid cage, or through substitution of methylene (CH₂) or methine (CH) positions in the diamondoid cage by heteroatoms such as nitrogen, oxygen, sulfur, boron, and phosphorous. As noted previously, such substitution is described in U.S. Pat. No. 7,049,374; and U.S. Patent Publications 2003/0199710 and 2002/0177743.

Table 1 below exemplifies the theoretical impact of various functional groups on the HOMO-LUMO gap of 4,9-disubstituted diamantane as a model compound for higher diamondoids. The effects are strong and are likely to translate to the ability to tune the fluorescence properties of diamondoids by selective functional group substitution.

The above-mentioned functionalization strategy builds on the idea that natural diamond also shows fluorescence and that this effect is due to microimpurities (i.e., atoms and groups other than carbon and hydrogen) that generate local polarities and as such affect the band structure.

The strategy of external and internal doping goes far beyond applications in lighting (electroluminescence and fluorescence) and should have implications for electronic building blocks as well. One could envision preparing n- and p-type substituted diamondoids that could be put together in electronic devices (diodes, transistors, field emitters, etc.).

TABLE 1

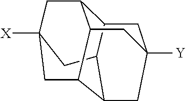

HOMO-LUMO gaps (energy separations, gas phase) for 4,9-
disubstituted diamantane derivatives as a model for higher diamondoids at
B3PW9I/6-31G(d, p) (1 eV = 23.06 kcal/mol)

| | | | | HOMO- | HOMO- |
|---|---|---|---|---|---|
| | | HOMO, - | | LUMO gap, | LUMO |
| X | Y | au | LUMO, au | kcal/mol | gap, eV |
| CF₃ | CF₃ | 0.28682 | 0.06085 | 218.2 | 9.46 |
| CH₃ | CF₃ | 0.27507 | 0.06753 | 215.0 | 9.32 |
| CH₃ | CH₃ | 0.26302 | 0.07463 | 211.9 | 9.19 |
| CH₃ | F | 0.27167 | 0.06354 | 210.3 | 9.12 |
| H | CH₃ | 0.26067 | 0.07362 | 209.8 | 9.10 |
| F | F | 0.27928 | 0.05393 | 209.1 | 9.07 |
| H | H | 0.25845 | 0.07273 | 207.8 | 9.01 |
| CN | CN | 0.29816 | 0.03153 | 206.9 | 8.97 |
| CH₃ | CN | 0.28111 | 0.04421 | 204.1 | 8.85 |
| OH | OH | 0.25278 | 0.06555 | 199.7 | 8.66 |
| CH₃ | NH₂ | 0.22692 | 0.07326 | 188.4 | 8.17 |
| NH₂ | NH₂ | 0.22738 | 0.07220 | 187.9 | 8.15 |
| CH═CH₂ | F | 0.25199 | 0.01364 | 166.7 | 7.23 |
| H | NH₃⁺ | 0.39239 | −0.13090 | 164.1 | 7.11 |
| NH₂ | COOH | 0.23055 | 0.01143 | 151.8 | 6.58 |
| Ph | CN | 0.24506 | −0.00602 | 150.0 | 6.50 |
| Ph | F | 0.24084 | −0.00212 | 149.8 | 6.50 |
| NO₂ | NO₂ | 0.29143 | −0.06691 | 140.9 | 6.11 |
| CH₃ | NO₂ | 0.27895 | −0.05669 | 139.5 | 6.04 |
| H | NO₂ | 0.27789 | −0.05655 | 138.9 | 6.02 |
| NH₃⁺ | COO⁻ | 0.11669 | −0.05125 | 41.1 | 1.78 |

Further examples of suitable candidates for tuned fluorescent diamondoid molecules are show in Scheme 1 below.

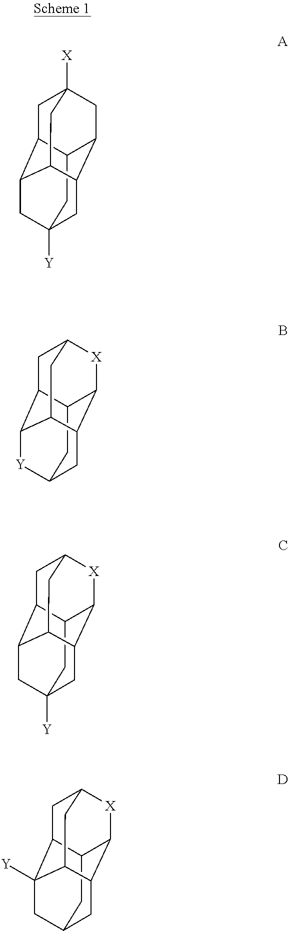

As noted above the C—H-bond functionalization with appropriate functional groups X and Y (for a selected but nonlimiting list see Table I) should allow for fine-tuning of the HOMO-LUMO gap and thus the fluorescence properties of diamondoids (Scheme 1 uses diamantane as an example for higher diamondoids as well). Electron-acceptor groups such as nitro and carboxy seem to be most promising to reduce the energy required to invoke fluorescence; push-pull systems appear to maximize this effect (e.g., the 4,9-amino acid, Table 1). It seems essential for the functional groups to be positioned at opposite ends for this strong polarization effect (A). Alternatively Y can also provide additional functionality for surface attachment (thiol, olefin, alkyne, hydroxy, acrylate, etc.).

The strategy of internal doping encompasses the incorporation of heteroatoms into the diamondoid cage (e.g., B above in Scheme 1). This is akin to doped diamonds as well as many other materials. Incorporation of heterofunctionality into the cage (internal doping) narrows the band gap strongly. See Table 2 below. The advantage over compound mixtures is, however, that such pure such materials can be produced. It is likely that compounds with X and/or Y═O, S, NR, BR, PR could be synthetically accessible; compound B has been prepared with X═O, Y═CH₂ and this is a stable compound.

Finally, external and internal can be combined in one structure such as in C and D (other substitution patterns are also possible), affording an even broader range of tunability.

TABLE 2

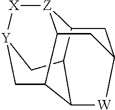

HOMO-LUMO gaps (energy separations, gas phase) for internally
doped diamantane as a model for higher diamondoids at
B3PW9I/6-31G(d, p) (1 eV = 23.06 kcal/mol)

| | | | | | | HOMO- | HOMO- |
|---|---|---|---|---|---|---|---|
| | | | | | | LUMO | LUMO |
| | | | | HOMO, - | | gap, | gap. |
| X | Y | Z | W | au | LUMO, au | kcal/mol | eV |
| CH₂ | CH | CH | CH₂ | 0.25845 | 0.07273 | 207.8 | 9.01 |
| O | CH | CH | CH₂ | 0.23035 | 0.07181 | 189.6 | 8.22 |
| S | CH | CH | CH₂ | 0.21083 | 0.03945 | 157.0 | 6.81 |
| NH | CH | CH | CH₂ | 0.20722 | 0.07141 | 174.8 | 7.58 |
| CH₂ | N | CH | CH₂ | 0.17345 | −0.08688 | 54.3 | 2.35 |
| CH₂ | CH | N | CH₂ | 0.18279 | −0.07153 | 69.8 | 3.03 |
| O | CH | CH | O | 0.23064 | 0.07515 | 191.9 | 8.32 |
| S | CH | CH | S | 0.20946 | 0.03453 | 153.1 | 6.64 |
| O | CH | CH | S | 0.21510 | 0.03586 | 157.5 | 6.83 |

The combination of visible light acceptors (such as aromatics) and frequency shifters (diamondoid fluorescence) should lead to light-harvesting devices that could be employed for increasing the efficiency of solar cells. A large variety of such compounds can be envisioned; two prototypical examples are depicted in Scheme 2 below. Both types of molecules can readily be made.

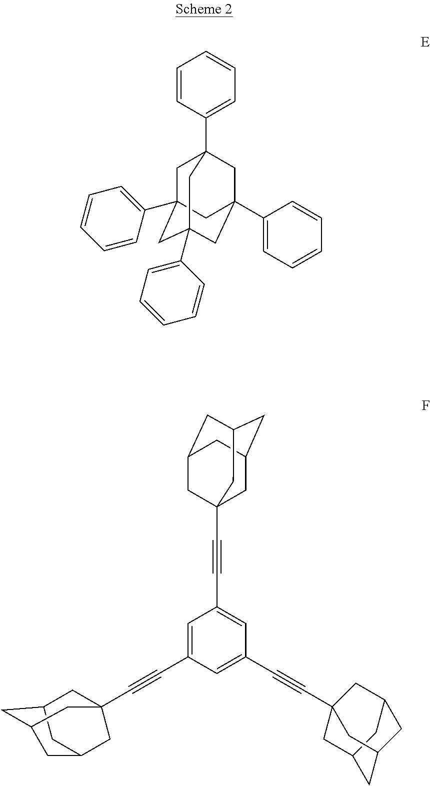

### Example

In a proof of principle experiment, the fluorescence spectra of different diamondoid crystals were measured. Adamantane, diamantane, and triamantane were measured, with the results shown in FIG. 3. [121] Tetramantane, and [1(2,3)4] pentamantane were measured with the results shown in FIG. 4. All measurements were made under 229 nm UV Illumination. Strong fluorescence spectra at visible light range (400-750 nm) were observed from all the diamondoid crystals.

Many modifications of the exemplary embodiments of the subject matter disclosed above will readily occur to those skilled in the art. Accordingly, the invention is to be construed as including all embodiments that fall within the scope of the appended claims.

1. A fluorescent diamondoid film comprised of a diamondoid, which film is situated in an environment to be energized and emit light once energized.

2. The diamondoid film of claim 1, wherein the film is in an environment to be energized by an electric field, high voltage or by high energy radiation.

3. The diamondoid film of claim 1, wherein the film is part of a solar cell.

4. The diamondoid film of claim 1, wherein the film is part of a lighting system.

5. The diamondoid film of claim 1, wherein the film emits light in the visible spectrum.

6. The diamondoid film of claim 1, wherein the film is in an environment to be energized by receiving a high energy photon and the film converts a single high energy photon into one or more lower energy visible photons and emits the visible light.

7. The diamondoid film of claim 6, wherein the high energy photon is a photon of UV light.

8. The diamondoid film of claim 1, wherein the diamondoid film is comprised of a diamondoid selected from the group of consisting of a higher diamondoid, lower diamondoid, functionalized diamondoid, heterodiamondoid, and mixtures thereof.

9. The diamondoid film of claim 8, wherein the diamondoid film is comprised of a mixture of diamondoids.

10. The diamondoid film of claim 8, wherein the diamondoid film is comprised of a functionalized diamondoid with the functional groups being chosen to fine tune the fluorescence shift of the film.

11. (canceled)

12. A solar cell comprised of a solar cell for collecting ultraviolet photons having a diamondoid layer on the surface facing the ultraviolet photons to be collected.

13. (canceled)

14. The solar cell of claim 12, wherein the diamondoid layer converts an ultraviolet photon into one or more visible photons.

15. The solar cell of claim 12, wherein the diamondoid material re-emits light at a wavelength which closely aligns with the absorption of the solar cell materials.

16. A solar cell comprised of a solar cell for collecting ultraviolet photons and further comprising a mixture of diamondoid material and semiconductor material with the diamondoid material being dispersed within the semiconductor material.

17. (canceled)

18. The solar cell of claim 16, wherein the diamondoid layer converts an ultraviolet photon into one more visible photons.

19. The solar cell of claim 16, wherein the diamondoid material re-emits light at a wavelength which closely aligns with the absorption of the solar cell materials.

20. A lighting system comprised of a fluorescent diamondoid film which is energized by an electric field to emit light.

21. The lighting system of claim 18, wherein light in the visible range is emitted.

22. A lighting system comprised of a fluorescent diamondoid film which is energized by high energy radiation to emit light in the visible range.

23. The lighting system of claim 20, wherein the diamondoid film is part of an organic light emitting diode.

24. (canceled)

25. (canceled)

26. (canceled)
